SEQUENCE LISTING (6 sequences)

SEQ ID NO: 1 - protein, 454 aa, Artificial
  Heavy chain
EVQLLESGGGLVQPGGSLRLSCAASGFTFSDYYMYWVRQAPEKRLEWVAYISNGGGSSHYVDSVKGRFTISRDNAKNTLYLQMNSLRAEDTALYHCARGMDYGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLHGDVSLTCLVKGFYPSDIAVEWESNGQPENNYKTTPPVLDSDGSFFLYSKLTVARYSPRMLRWAHGNVFSCSVMHEALHNHYTQKSLSLSPGK

SEQ ID NO: 2 - protein, 219 aa, artificial
  Light chain
DIVMTQSPLSLPVTPGEPASISCRSSQSIVHSNGNTYLEWYLQKPGQSPQLLISKVSNRFSGVPDRFSGSGSGTDFTLKISRVEAEDVGVYYCFQGSHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, artificial
  heavy chain
EVQLLESGGGLVQPGGSLRLSCAASGFTFSDYYMYWVRQAPEKRLEWVAYISNGGGSSHYVDSVKGRFTISRDNAKNTLYLQMNSLRAEDTALYHCARGMDYGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEFFTYWVSLTCLVKGFYPSDIAVEWESNGQPENNYKTTPPVLDSDGSFFLYSKLTVDRRRWTAGNVFSCSVMHEALHNHYTQKSLSLSPGK

SEQ ID NO: 4 - protein, 219 aa, artificial; identical to SEQ ID NO: 2
  light chain

SEQ ID NO: 5 - protein, 19 aa, artificial
  signal peptide
MELGLSWIFLLAILKGVQC

SEQ ID NO: 6 - protein, 22 aa, artificial
  signal peptide
MDMRVPAQLLGLLLLWLPGAKC

(19) United States
(12) Patent Application Publication
(10) Pub. No.: US 2012/0010388 A1
(43) Pub. Date: Jan. 12, 2012

(54) LeY SPECIFIC BIOTHERAPEUTIC

(76) Inventor: Gottfried HIMMLER, Gross-Enzersdorf (AT)

(21) Appl. No.: 13/228,559

(22) Filed: Sep. 9, 2011

Related U.S. Application Data
(63) Continuation of application No. 13/086,897, filed on Apr. 14, 2011, now abandoned.

(30) Foreign Application Priority Data
Apr. 16, 2010 (EP) EP10160142

(51) Int. Cl. C07K 16/30

(52) U.S. Cl. 530/387.3

(57) ABSTRACT

Embodiments are related to the field of immunology, and provide a highly avid LeY specific biotherapeutic including at least one further binding site having a different specificity to bind an epitope of a glycosylated cell surface molecule of a tumor cell, characterized by EC50 of less than 1 mM to confer immediate cytotoxicity to the tumor cell.

### CROSS REFERENCE TO PRIOR APPLICATIONS

The present application is a continuation application of U.S. patent application Ser. No. 13/086,897, which claims priority under 35 U.S.C. §119 to European Patent Application No. EP10160142 (filed on Apr. 16, 2010), each of which is hereby incorporated by reference in there complete entireties.

### FIELD OF THE INVENTION

The invention relates to a cytotoxic LeY-specific biotherapeutic. Cancer immunotherapy typically targets cell surface molecules, that are overexpressed in tumor cells. Tyrosine kinase receptors, e.g. those of the erbB family are common targets of antibody therapy (such as members of the EGFR family, of the insulin receptor family, of the PDGF family, of the VEGF receptor family, of the HGF receptor family.

### BACKGROUND OF THE INVENTION

Receptor tyrosine kinases (RTK)s are the high-affinity cell surface receptors for many polypeptide growth factors, cytokines, and hormones. Receptor tyrosine kinases have been shown not only to be key regulators of normal cellular processes but also to have a critical role in the development and progression of many types of cancer.

Most RTKs are single subunit receptors but some exist as multimeric complexes, e.g., the insulin receptor that forms disulfide-linked dimers in the absence of hormone; moreover, ligand binding to the extracellular domain induces formation of receptor dimers. Each monomer has a single hydrophobic transmembrane-spanning domain composed of 25-38 amino acids, an extracellular N-terminal region, and an intracellular C-terminal region. The extracellular N-terminal region exhibits a variety of conserved elements including immunoglobulin (Ig)-like or epidermal growth factor (EGF)-like domains, fibronectin type III repeats, or cysteine-rich regions that are characteristic for each subfamily of RTKs; these domains contain primarily a ligand-binding site, which binds extracellular ligands, e.g., a particular growth factor or hormone. The intracellular C-terminal region displays the highest level of conservation and comprises catalytic domains responsible for the kinase activity of these receptors, which catalyses receptor autophosphorylation and tyrosine phosphorylation of RTK substrates.

The ErbB protein family or epidermal growth factor receptor (EGFR) family is a family of four structurally related receptor tyrosine kinases. Excessive ErbB signaling is associated with the development of a wide variety of types of solid tumor. ErbB-1 and ErbB-2 are found in many human cancers and their excessive signaling may be critical factors in the development and malignancy of these tumors.

The fibroblast growth factor receptors are receptors (FGFR) which bind to members of the fibroblast growth factor family of proteins. The natural alternate splicing of four fibroblast growth factor receptor genes results in the production of over 48 different isoforms of FGFR.

Vascular endothelial growth factor (VEGF) is one of the main inducers of endothelial cell proliferation and permeability of blood vessels. The VEGF receptors have an extracellular portion consisting of seven Ig-like domains so, like FGFRs, belong to the immunoglobulin superfamily.

Eph receptors are components of cell signaling pathways involved in animal development, and implicated in some cancers. Eph receptors are classified as receptor tyrosine kinases (RTKs), and form the largest sub-family of RTKs.

RET receptor family:activating point mutations in RET can give rise to the hereditary cancer syndrome known as multiple endocrine neoplasia type 2.

One of the most prominent representative of a Tyrosine Kinase Receptor as a biotherapeutic target is HER2.

HER2 (also known as ErbB-2, CD340, p185) is a member of the ErbB protein family and is a cell membrane surface-bound receptor tyrosine kinase, normally involved in the signal transduction pathways leading to cell growth and differentiation. It is encoded within the genome by HER2/neu.

Approximately 15-20 percent of breast cancers have an amplification of the HER2/neu gene or overexpression of its protein product. Overexpression also occurs in other cancer such as ovarian cancer, stomach cancer, and biologically aggressive forms of uterine cancer, such as uterine serous endometrial carcinoma. HER2/neu is important as the target of the monoclonal antibody trastuzumab (Herceptin). Overexpression of the HER2 gene can be suppressed by the amplification of other genes and the use of Herceptin.

Although trastuzumab is generally well tolerated, cardiac toxicity has emerged as a potentially serious complication that limits its use in some patients, particularly when given in combination with anthracyclins. HER2 is expressed on cardiomyocytes, in addition to tumor tissue, where it exerts a protective effect on cardiac function; thus, interference with HER2-signaling may block this protective effect.

The majority of trastuzumab-related cardiac events observed have been asymptomatic declines in left ventricular ejection fraction. The incidence of severe congestive heart failure and cardiac death observed in the large adjuvant trastuzumab trials ranges from 0.6 to 4%. Approximately one in four breast cancer patients undergoing anthracycline-based chemotherapy with subequent trastuzumab use may require discontinuation of the drug as a result of cardiotoxicity.

Another monoclonal antibody binding to HER2, Pertuzumab, which inhibits dimerization of HER2 and HER3 receptors, is in advanced clinical trials. Cardiac toxicity has also been observed for pertuzumab treatment.

Close cardiac monitoring must be performed for all patients receiving anti-HER2 agents currently in the clinic or in development. Such side effect may be enhanced by using more effective therapeutics, such as antibodies with increased ADCC potency.

Although in clinical trials cardiac toxicity related to trastuzumab did not seem to be dose dependent, there might be a threshold for this toxic effect. Consequent lowering of the dose of trastuzumab may significantly decrease incidents of cardiac toxicity. This may however also abolish the therapeutic effect of the antibody. It would therefore be useful to restore at such low doses the therapeutic potential of the anti-HER2 agent by another binding site for another tumor associated antigen.

Overexpression of HER2 in only a third of breast cancer patients limits the use of anti-HER2 agents. Expression and co-targeting of other tumor associated antigens than HER2 on tumor cells might be useful to define a broader range of patients amenable to HER-specific therapy.

Another prominent example of receptor tyrosine kinases is EGFR. The epidermal growth factor receptor (EGFR; ErbB-1; HER1 in humans) is the cell-surface receptor for members of the epidermal growth factor family (EGF-family) of extracellular protein ligands. The epidermal growth factor receptor is a member of the ErbB family of receptors. Mutations affecting EGFR expression or activity can result in cancer. Antibodies against EGFR are in clinical use for various cancers. Due to expression of EGFR on normal epithelium frequent side effects are associated with this treatment. Depending on the particular EGFR inhibitor, an acneiform rash occurs in 30 to 90% of patients.

Several types of mutations have been observed to confer resistance to anti-EGFR antibodies (such as KRAS mutations, MKP-1 overexpression and the like).

### Lewis Y (LeY)

Lewis Y (CD174), is a difucosylated disachcharide antigen (Fuc-alpha-(1-2)-Gal-beta(1-4)-Fuc-alpha-(1-3)-GlcNAc-beta-1-) detected and characterized originally on epithelial cells or in body fluids and secretions. LeY expression has been observed in normal colonic mucosa (terminal ileum, cecum, and ascending colon and minimal expression in the rest of the colon and rectum).

The normal processes of glycosylation are often severely altered during oncogenic transformation of animal cells.

The majority of cancer cells derived from epithelial tissue (including breast, ovary, pancreas, prostate, colon and non-small cell lung cancers) express Lewis-Y (LeY) type difucosylated oligosaccharides, either at the plasma membrane as a glycolipid or linked to surface receptors (e.g. of the ErbB family).

LeY expression has been observed in normal colonic mucosa (terminal ileum, cecum, and ascending colon and minimal expression in the rest of the colon and rectum). The normal processes of glycosylation are often severely altered during oncogenic transformation of animal cells or infections with some viruses. The majority of cancer cells derived from epithelial tissue (including breast, ovary, pancreas, prostate, colon and non-small cell lung cancers) express Lewis-Y (LeY) type difucosylated oligosaccharides, either at the plasma membrane as a glycolipid or linked to surface proteins.

Lewis Y can be up-regulated in response to cellular stress. Up-regulation of Lewis Y in response to chemotherapeutic stress has been observed. In early stage breast cancer, expression of Lewis Y may, therefore, be a marker of aggressive or stressed tumours.

Lewis Y specific treatments of various carcinomas have been reported. Some side effects (gastrointestinal toxicity) observed can be attributed to the expression of LewisY on normal tissue such as cells of the gastric tract. Some side effects of anti-Lewis Y antibodies can be attributed to the frequent crossreactivity of such anti-Lewis Y antibodies with other blood group related carbohydrate motifs such as Lewis X or H-type2.

The cytotoxic effect of antibodies is mainly based on their effector functions, as measured in either an ADCC or CDC assay. Antibody-Dependent Cell-Mediated Cytotoxicity (ADCC) is a mechanism of cell-mediated immunity, whereby an effector cell of the immune system actively lyses a target cell that has been bound by specific antibodies. It is one of the mechanisms through which antibodies, as part of the humoral immune response, can act to limit and contain infection. Classical ADCC is mediated by natural killer (NK) cells. Complement-dependent cytotoxicity (CDC) is mediated by the complement system.

WO 2009/132876 describes small sized cytotoxic anti-Her2 immunoglobulins with a high binding affinity and cytotoxic activity, in particular as measured by an ADCC test. Bispecific immuntherapeutics have been developed to improve the specificity of treatment.

WO 1994024163 describes methods of restricting cell growth and treating disease using molecules that inhibit cellular function. In particular, the disclosure relates to methods of inhibiting cellular function with an “antigen fork”, like a bispecific antibody possessing two separate binding elements, where one binding element recognizes a different cell surface antigen than a second binding element, and where the two different antigens have distinct functional properties.

EP468637A1 provides bispecific antibodies (Babs) and immuno-conjugates to achieve decreased cross-reactivity with critical normal tissue while maintaining avid target specificity. The molecules would be able to selectively direct a wide variety of agents to particular sites in the body for an assortment of purposes while reducing the level of cross-reactivity to undesired locations.

WO1997008205 concerns antibodies having two or more specificities detecting two different antigens localized on a tumour cell. A series of targets are described to produce bispecific antibodies to specifically recognize and eliminate cancer by ADCC, amongst others the combination of anti-Lewis Y/anti-c-erb-B2. The antigen Lewis Y is referred to as described in by Pastan et al (1991, Cancer Research, volume 51, pages 3781ff), which disclosed LeY antibodies cross-reactive with Lewis X antigen and the H2-type antigen.

Thomas Kieber-Emmons in a report to the US Army Medical Research & Materiel Command (Report Number B222453; 1996; US Army Med Research and Mat Cmd, MCMR-RMI-S [70-1y], ltr 6 Jul. 2000, Ft Detrick, Md.) mentions the chemical crosslinking of two antibodies, an anti-EGFR and an anti-Lewis Y antibody. He showed that an increased efficacy of binding on target cells can be accomplished when compared to binding of the anti-Lewis Y antibody alone. The chemically crosslinked bispecific antibodies in this report did not show an immediate cell killing effect.

WO2005117973 and WO2006091209 describe bispecific binding agents that are able to target cells by a high affinity binding domain to a first cell surface marker that does not induce a significant biological effect and a low affinity binding domain that binds specifically to a second cell surface marker, causing a significant and desired biological effect.

WO2005/014618 A2 describes an apoptosis-inducing heteroconjugate that comprises two or more different species of antibodies binding the antigens via CDR-binding sites.

WO2008003103 describes multivalent immunoglobulins or parts thereof binding specifically to at least two cell surface molecules of a single cell with at least one modification in at least one structural loop region of said immunoglobulin determining binding to an epitope of said cell surface molecules wherein the unmodified immunoglobulin does not significantly bind to said epitope, its use and methods for producing it.

U.S. Patent Publication No. 2005/018147 describes the combination of vaccine antigens.

### DESCRIPTION

There is a need to overcome the limited efficacy of immunotherapy that targets cell surface molecules of tumor cells, in particular of tyrosine kinase receptors, such as HER2, while preventing potential side effects.

The present invention overcomes these problems by combining a specifc anti-LeY binding site that would not cross-react with other carbohydrate moieties with a site binding to a cell surface target on a tumor cell in one molecule having a high avidity to confer immediate and direct cytotoxicity, without the need to rely on mediator such as NK cells or complement factors.

A bispecific molecule binding to a carbohydrate motif, such as LeY, which is on the surface of a tumor cell and a tumor associated surface protein of a tumor cell may kill a tumor cell immediately, if there is a high avidity. For example, the anti-Lewis Y antibody IGN311 (humanized ABL364, Co et al, Cancer Research, 1996 Mar. 1; 56(5):1118-25) was used to graft a HER-2 binding site onto it by using an anti-HER-2 Fcab molecule, such as described in WO 2009/132876. An Fcab molecule (“Fc antigen binding”) consists of an antibody Fc fragment with an antigen-binding site engineered into the structural loops. It has been surprisingly found that such a molecule kills HER-2 positive/Lewis Y positive cells immediately and much stronger than either the anti-Lewis antibody or the anti-HER2 Fcab alone. This cytotoxic effect is much stronger than the effect measured in a typical ADCC assay. In fact, there was no measurable ADCC during the cell killing assay, because cells were immediately killed already before adding effector (NK) cells. There was a synergistic effect of binding sites proven with those cells that express both, the carbohydrate epitope and the tumor associated surface protein. Normal cells expressing only one of the antigens would not be immediately killed by the biotherapeutic molecule according to the invention.

The same effect may be observed with other constructs targeting at least any combination of LeY and a tumor associated surface protein.

As surface protein structures basically any cell surface protein can be targeted. Preferentially one embodiment targets tumor associated proteins, e.g., members of the erb-family of proteins or cell adhesion molecules or other receptors on the cell surface.

The advantage of such a bispecific molecule would be on the one hand enhanced efficacy with a synergistic effect. On the other hand a therapeutic molecule may be engineered with the respective binding sites having low affinity to normal cells and a higher tumor selectivity. Thus, the bispecific molecule would not bind to a cell expressing only one of the two targets, which may be the case with non-tumor cells, but would only bind strongly to a cell expressing both targets, which would be a tumor cell. Co-expression of various tumor targets, such as combinations of the LeY carbohydrate and protein antigens, has been shown to be of worse prognosis.

One could also envisage more than two targets for such a molecule (e.g. two different carbohydrate antigens and one protein antigen, two protein antigens and one carbohydrate etc.).

Besides the immediate cytotoxicity eventually effector functions and long half life may be desired or not, depending on the desired effect.

The molecule may not necessarily be an antibody derivative but could also be alternative scaffold binders or even a combination of different binders, and need not even to be proteins (could be e.g. aptamers or small molecules).

To allow for a wide range of possible applications, the biotherapeutic according to the invention would mediate its cytotoxic effect without the dependence on further components of the immune system. This is particularly useful, because most patients receive for the treatment of, e.g. cancer, standard chemo- or radiotherapy. Most of these treatments leave the patient immunocompromised, which could also damage the effect of mediators of cytotoxicity. Thus, the biotherapeutic according to the invention is particularly suitable for combination therapy with chemo- or radiation therapy.

Since the biotherapeutic according to the invention is highly avid, the killing on the target cells is quickly effected, even with low doses.

The highly avid LeY specific biotherapeutic according to the invention is having at least one further binding site having a different specificity to bind an epitope of a glycosylated cell surface molecule of a tumor cell, and is characterized by an EC50 of less than 1 mM, preferably less than 10⁻⁴ M, more preferably less than 10⁻⁵ M, more preferably less than 10⁻⁶ M, more preferably less than 10⁻⁷ M, more preferably less than 10⁻⁸ M, more preferably less than 10⁻⁹ M, more preferably less than 10⁻¹⁰ M, even in the picomolar range, to confer immediate cytotoxicity to said diseased mammalian cell.

A biotherapeutic is a molecule of peptidic, polypeptidic or proteinaceous origin, preferably human or for human use, which is used for specific therapeutic purposes. It may consist of one or more polypeptide chains. The molecule may be modified to also contain non-peptidic motifs. Such a modification may e.g. be a natural post-translational modification such as glycosylation or an artificial modification such as chemical covalent or non-covalent coupling of non-peptidic motifs (e.g. coupling of PEG or radiolabels etc.).

Typically a biotherapeutic is a therapeutic drug based on a biological rather than a small molecule. A biotherapeutic according to the invention may act like a chemotherapeutic, but is based on a biological substance.

Binding site means a motif of a molecule of the invention which makes contact to a specific epitope.

The term “epitope” means a target structure on a chemical entity which can be recognized by a binding molecule to be different from most other structures. An epitope can be a peptide or part of it, a carbohydrate structure or other chemical entities (such as lipids, phosphorylation sites) or a combination of more than one chemical entity. An epitope can also be formed by certain spatial configurations of chemical structures in which different motifs from various parts of a molecule or molecule complex are in close spatial vicinity.

A glycosylated cell surface molecule is a molecule displayed on the surface of a target cell, which has at least one carbohydrate motif associated with it.

A target cell as meant in the context of this invention is a tumor cell displaying at least Lewis Y carbohydrate motifs and a specific glycosylated surface molecule.

Immediate cytotoxicity as defined in the context of this invention means the cytotoxicity conferred to target cells by a molecule without the help of mediators or other factors such as effector cells or complement. It is measured by quantitating the living and/or dead target cells before and after addition of the molecule to target cells. The cytotoxicity is expressed in the distribution or share of dead cells after a certain period under certain conditions of incubation with the molecule starting with a cell population of 100% living cells. A preferred method of determining immediate cytotoxicity comprises incubating cells from a cancer cell line with a series of dilutions of the test molecule in a cell culture medium for a short period of time, such as 10 min or 15 min. Then the distribution of dead and viable cells is immediately measured. A test molecule having a direct or immediate cytotoxic effect on such cancer cells would typically lead to the cell death of at least 20%. This cytotoxic effect is increased with molecules that bind to the target cell with a high avidity, increasing with the number of binding sites. Such an immediate cytotoxic effect has not been described with immunotherapeutics targeting tumor associated antigens. It was surprising that this effect was found with the combination of binding to the LeY antigen (without cross-reactivity to LeX or other blood-group related carbohydrates) and binding to other epitopes of the same or adjacent molecules on the surface of a tumor cell, even though an immunotherapy targeting the individual antigens would not bring about such an effect.

EC50 in the context of this invention means the concentration of the molecule of the invention which gives the half maximal signal in a cell killing or cell binding assay. The EC50 for binding is determined, for example by reacting a dilution series of the molecule with target cells and subsequent detection of bound molecules on the cell surface. The detection can be performed in a FACS analyzer. The EC50 for cytotoxicity can be determined e.g. by incubating a certain number of living target cells with the molecule of the invention in various concentrations for a certain period of time and measuring the number of dead cells at the end of this period. The percentage of dead or apoptotic cells for each concentration of the biotherapeutic is plotted against the concentration. The concentration which resulted in 50% dead cells is the EC50. Usually various controls can be incorporated in such assays such as incubation with an agent which kills cells fast and completely to give a 100% cell killing value.

The measurement can be based on assaying the cell membrane integrity such as the chromium-release assay, assays based on trypan blue or propidium iodide staining. Alternatively, membrane integrity can be assessed by monitoring the passage of substances that are normally sequestered inside cells to the outside. One commonly measured molecule is lactate dehydrogenase (LDH). Protease biomarkers have been identified that allow researchers to measure relative numbers of live and dead cells within the same cell population. The live-cell protease is only active in cells that have a healthy cell membrane, and loses activity once the cell is compromised and the protease is exposed to the external environment. The dead-cell protease cannot cross the cell membrane, and can only be measured in culture media after cells have lost their membrane integrity.

Cytotoxicity can also be monitored using the MTT or MTS assay. This assay measures the reducing potential of the cell using a colorimetric reaction. Viable cells will reduce the MTS reagent to a colored formazan product. A similar redox-based assay uses the fluorescent dye resazurin. Assays that use ATP content as a marker of viability can also be used.

Cytotoxicity can also be measured by the sulforhodamine B (SRB) assay, WST assay and clonogenic assay.

A label-free approach to follow the cytotoxic response of adherent animal cells in real-time is based on electric impedance measurements when the cells are grown on gold-film electrodes. This technology is referred to as electric cell-substrate impedance sensing (ECIS).

In addition to assessment of membrane integrity one can also use assays for apoptosis. All these assays are examples and there can be many different variations and combinations to it.

To be tested is for inhibition or prevention of the function of a cell and/or destruction of a cell. Most preferably one uses a combination assays that are indicating the degree of necrosis plus the degree of apoptosis.

Specifically, the biotherapeutic according to the invention is characterized by an immediate cytotoxicity determined in an in vitro assay comprising incubating said tumor cell with the biotherapeutic as the only active ingredient. In one example the cytotoxicity of a biotherapeutic of the invention is measured by a chromium release assay. The target tumor cells are labelled with ⁵¹Cr, then incubated with the biotherapeutic in various concentrations for a short period of time and the released ⁵¹Cr in the supernatant is measured. A maximum release of ⁵¹Cr is achieved by treating the cells with a detergent (100%). The ⁵¹Cr release values resulting from the various concetrations of the biotherapeutic are plotted against the concentration of the biotherapeutic. The concentration which yields 50% of ⁵¹Cr-release after 1 hour is the EC50.

Avidity is a measure for the strength of binding of a molecule to target structures. Avidity to a target cell is a function of all the affinities of individual binding sites of a molecule of the invention to both Lewis Y and the specific glycoprotein epitope on a target cell. Specifically the avidity is more than the sum of the individual affinies of binding sites of a molecule. Avidity can be measured by various methods such as equlibrium or non-equlibrium techniques. In a preferred technique for measurement of avidity of a molecule to a cell, dilution experiments and homogenous competition curves (i.e., competition for cell binding between the labeled and unlabeled forms of the same molecule of the invention) are analyzed by a linear equilibrium model. The free (F) and bound (B) label are measured and Kd is calculated according to the Scatchard equation.

Typically the biotherapeutic according to the invention is considered a highly avid molecule, with a fast on-rate kinetics.

A biotherapeutic according to the invention is preferably used, which has a low binding affinity to normal cells with a Kd of more than 1 mM, preferably more than 10⁻² M.

In a preferred embodiment the biotherapeutic according to the invention has a binding affinity to a target tumor cell with a Kd in the range of 10⁻⁶ M to 10⁻¹² M, preferably the binding affinity to either of the LeY or the other cell surface epitopes is relatively low, with a Kd of more than 10⁻⁹, preferably more than 10⁻⁸ M, more preferred of more than 10⁻⁷ M. This individual binding affinity is typically measured in a Biacore test system with the immobilized antigens. Individual binding affinities may differ from each other, e.g. such that the binding affinity to the LeY antigen is less than the binding affinity to the other epitope(s) of the bi- or multispecific construct. However, the total binding affinity to all relevant epitopes of a target cell or in a Biacore test system with isolated antigens (avidity) would be high, particularly at least 10 fold increased to the highest individual binding affinity, preferably at least 100 fold increased, more preferably at least 1000 fold increased.

The term “immediate cytotoxicity” means cytotoxicity induced by a molecule of the invention without additional factors such as complement or effector cells. This immediate cytotoxicity may translate to necrosis of cells or early or late apoptosis of cells. The immediate cytotoxicity can be measured typically relatively fast after addition of a biotherapeutic of the invention in contrast to growth inhibition of cells. It can be measured after 10 min to 6 hours after addition of substance to cells.

A tyrosine kinase receptor is the extracellular part of a receptor tyrosine kinase. Currently known classes of receptor tyrosine kinases are: RTK class I (EGF receptor family) (ErbB family), RTK class II (Insulin receptor family), RTK class III (PDGF receptor family), RTK class IV (FGF receptor family), RTK class V (VEGF receptors family), RTK class VI (HGF receptor family), RTK class VII (Trk receptor family), RTK class VIII (Eph receptor family), RTK class IX (AXL receptor family), RTK class X (LTK receptor family), RTK class XI (TIE receptor family), RTK class XII (ROR receptor family), RTK class XIII (DDR receptor family), RTK class XIV (RET receptor family), RTK class XV (KLG receptor family), RTK class XVI (RYK receptor family), RTK class XVII (MuSK receptor family).

A target tumor cell is, for example, a solid tumor cell, such as an epithelial tumor cell, that would express both targets LeY and the glycosylated cell surface molecule. The LeY carbohydrate moiety preferably is part of the aberrant glycosylation of said cell surface molecule. Though the second specificity is different from the LeY specificity, it may target another carbohydrate epitope or a peptidic epitope or a structural (three-domensional) epitope of said cell surface protein. It is preferred that adjacent targets are combined, e.g. the combination of targets is located on a single cell surface molecule. Thus, a preferred biotherapeutic according to the invention binds to said cell surface molecule by its binding sites to LeY and said epitope.

In a preferred example, the biotherapeutic according to the invention targets an epitope, which is one of a tyrosine kinase receptor or a cell adhesion molecule that are usually overexpressed on the surface of tumor cells. Specifically a preferred epitope is one of an erbB molecule, selected from the group consisting of EGFR, HER2, HER3, HER4.

Preferred biotherapeutics are composed of immunoglobulins or immunoglobulin-like molecules. “Immunoglobulins” are understood as molecules comprising immunoglobulin-domains binding to specific epitopes. Examples of such immunoglobulins are antibodies and antibody fragments, immunoadhesins. Complete antibodies can be antibodies of all classes (e.g. IgG,IgA, IgD, IgM, IgE, IgY etc) and subclasses (e.g. IgG1, IgG2, IgG3, IgG4) and all species (e.g. human, primates, rodent such as mouse, rat, rabbit; shark, chicken etc.). Suitable antibody fragments can be e.g. a Fab, (Fab)₂, Fv, scFv, single domains, diabody, mini-antibody, Fcab, Tandab, di-diabody and the like, which may be designed to provide for multispecificity, including bispecific approaches.

Fusions of antibody fragments with each other or with complete antibodies are also considered immunoglobulins. The antibodies or antibody fragments may be modified either chemically or by recombinant DNA technologies to change the properties of such molecules (e.g. half life, effector functions such as antibody dependent cellular cytotoxicity and complement dependent cytotoxicity).

“Immunoglobulin-like molecules” are understood as molecules able to bind to specific epitopes. The molecules may occur naturally or be designed to select from libraries of molecules the ones with specific binding. Such molecules may be composed of immunoglobulin-like domains or other basic structures such as non-antibody scaffolds (examples are DARPins, Avimers, anticalins and the like, it might also be simply aptamers whether peptidic or not, as well as Fc fusions therof).

A biotherapeutic according to the invention, having at least 3 binding sites, preferably at least 4, 5, 6, or more, possibly up to 8 binding sites to said tumor cell is preferred to increase the avidity of the molecule. For example, molecules may be engineered that—in addition to the LeY binding site—target more than one epitope of one or more cell surface molecules. A further embodiment relates to more than one valencies to bind either of the LeY antigen or the cell surface antigen.

The biotherapeutic according to the invention typically would not act as a commonly used immunotherapeutic, because of its immediate cytotoxicity. Comparable to a chemotherapeutic its action is immediate. Due to the fast cell killing effect achieved according to the invention, the biotherapeutic would not rely on any ADCC or CDC activity. Thus, constructs lacking ADCC and/or CDC would be specific embodiments that would likewise show the immediate cytotoxic effect. On the other hand, antibody constructs are preferably used that comprise a functional immunoglobulin Fc fragment, which would naturally have an ADCC and/or CDC acitivity, not hindering the immediate cytotoxic effect through simultaneous binding and cross-linking of the target combinations according to the invention.

A biotherapeutic according to the invention is specifically preferred, which is a recombinant antibody with binding sites in the CDR and in the non-CDR region. The natural antigen-binding site of an antibody is formed by CDR loops of VH and/or VL domains. In addition to the VH/VL binding site, a further antigen binding site may be formed by peptides or antigen-binding domains fused to an antibody construct, or else formed by mutations of the antibody construct, which bring about a change in the amino acid sequence in the non-CDR region, thereby determining a new antigen binding sites such as pockets or other surfaces. A method of constructing recombinant antibodies in a modular way, with new additional binding sites in the structural loop region is described, e.g. by WO 2009/132876.

A preferred method of producing a biotherapeutic according to the invention includes the following steps: a) providing a biological with at least one binding site specifically binding to LeY; b) engineering said molecule to add at least one further binding site specifically binding to an epitope of a glycosylated cell surface molecule of a tumor cell, wherein said binding sites are designed to confer an avidity with an EC50 of less than 1 mM to kill a tumor cell, and c) formulating a pharmaceutically acceptable preparation.

There are a multitude of technologies to produce bispecific or multispecific molecules. Some of the technologies are in the field of polypeptides or proteins (such as immunoglobulins, antibodies or alternative scaffold binders) and are based on the fact that covalent or non-covalent association of such polypeptides with specific binding sites can be engineered. Examples are bispecific and multispecific antibodies or antibody fragments in the form of quadromas, diabodies, knob-into hole Fc bearing antibodies, SEED-bodies, fusions of antibody fragments to antibodies or Fc and the like. Alternative scaffolds have also been shown to provide for bispecific or multispecific biological molecules.

Various antigen- or ligand binding polypeptides can be fused genetically to form one polypeptide chain with more than one binding site, the various binding polypeptides may be linked with synthetic or natural linkers to provide optimal spacing and overall binding effects.

The polypeptides may also be chemically linked with reactive groups. Here also linker can be selected to provide optimal spatial configuration of the various binding sites.

The biotherapeutic according to the invention is specifically suitable for use as an anti-cancer drug. A specific embodiment refers to a method of producing a pharmaceutically acceptable preparation comprising a biotherapeutic according to the invention, for use as an anti-cancer drug. Due to the expected high efficacy of treatment the biotherapeutic according to the invention would be specifically used in anti-cancer monotherapy. Further, its use on top of standard therapy, e.g. in combination with chemotherapy and/or radiation is envisaged.

Particularly suitable therapy is directed against a cancer selected from the group of breast, gastric, colon, ovarian, lung, prostate cancer, germ cell tumors, esophageal tumors, head and neck cancers, bladder tumors, cervical cancer and renal cancer. Specific indications may also be other tumors such as sarcomas, leukemias, lymphomas and myelomas and tumors of the central nervous system.

The molecule of the invention may be formulated, dosed, and administered consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. The “therapeutically effective amount” of the molecules of the invention to be administered will be governed by such considerations, and is the minimum amount necessary to prevent, ameliorate, or treat a neoplastic disease. The biotherapeutic need not be, but is optionally, formulated with one or more agents currently used to prevent or treat a tumor or a risk of developing cancer. The effective amount of such other agents depends on the amount of molecule of the invention present in the formulation, the type of disorder or treatment, and other factors as discussed above. These are generally used in the same dosages and with administration routes as used hereinbefore or about from 1 to 99% of the heretofore employed dosages. Generally, alleviation or treatment of a cancer involves the lessening of one or more symptoms or medical problems associated with the cancer. The therapeutically effective amount of the drug can accomplish one or a combination of the following: reduce (by at least 10%, 20%, 30%, 40%, 50%, 60%, 70%, 80%, 90%, 100% or more) the number of cancer cells; reduce or inhibit the tumor size or tumor burden; inhibit (i.e., to decrease to some extent and/or stop) cancer cell infiltration into peripheral organs; reduce hormonal secretion in the case of adenomas; reduce vessel density; inhibit tumor metastasis; reduce or inhibit tumor growth; and/or relieve to some extent one or more of the symptoms associated with the cancer. In some embodiments, the molecule of the invention is used to prevent the occurrence or reoccurrence of cancer or an autoimmune disorder in the subject.

For the prevention or treatment of disease, the appropriate dosage of a molecule of the invention will depend on the type of disease to be treated, the severity and course of the disease, whether the molecule is administered for prophylaxis of recurrence or therapeutic purposes, previous therapy, the patient's clinical history and response to the biotherapeutic, and the discretion of the attending physician. The molecule is suitably administered to the patient at one time or over a series of treatments. Depending on the type and severity of the disease, about 1 μg/kg to 15 mg/kg (e.g. 0.1-20 mg/kg) of molecule is an initial candidate dosage for administration to the patient, whether, for example, by one or more separate administrations, or by continuous infusion. A typical daily dosage might range from about 1 μg/kg to 100 mg/kg or more, depending on the factors mentioned above. An exemplary dosage of the molecule of the invention to be administered to a patient is in the range of about 0.1 to about 10 mg/kg of patient weight.

For repeated administrations over several days or longer, depending on the condition, the treatment is sustained until a desired suppression of disease symptoms occurs. Other dosage regimens may be useful. The progress of this therapy is easily monitored by conventional techniques and assays.

In one embodiment, the present invention can be used for increasing the duration of survival of a human patient susceptible to or diagnosed with a cancer or other tumors. Duration of survival is defined as the time from first administration of the drug to death. Duration of survival can also be measured by stratified hazard ratio (HR) of the treatment group versus control group, which represents the risk of death for a patient during the treatment.

In yet another embodiment, the treatment of the present invention significantly increases response rate in a group of human patients susceptible to or diagnosed with a cancer or another tumor who are treated with various anti-tumor therapies. Response rate is defined as the percentage of treated patients who responded to the treatment. In one aspect, the combination treatment of the invention using a molecule of the invention and surgery, radiation therapy, or one or more chemotherapeutic agents significantly increases response rate in the treated patient group compared to the group treated with surgery, radiation therapy, or chemotherapy alone.

Therapeutic formulations are prepared using standard methods known in the art by mixing the active ingredient having the desired degree of purity with optional physiologically acceptable carriers, excipients or stabilizers (Remington's Pharmaceutical Sciences (20th edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

Sustained-release preparations may be prepared. Suitable examples of sustained-release preparations include semipermeable matrices of solid hydrophobic polymers containing the biotherapeutic, which matrices are in the form of shaped articles, e.g., films, or microcapsule. Examples of sustained-release matrices include polyesters, hydrogels, polylactides, copolymers of L-glutamic acid and γ ethyl-L-glutamate, non-degradable ethylene-vinyl acetate, degradable lactic acid-glycolic acid copolymers (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated molecules of the invention remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The molecules of the invention may be administered to a human subject according to known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Local administration may be particularly desired if extensive side effects or toxicity is observed. An ex vivo strategy can also be used for therapeutic applications. Ex vivo strategies involve transfecting or transducing cells obtained from the subject with a polynucleotide encoding a biotherapeutic. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells.

Another embodiment of the invention is an article of manufacture containing materials useful for the treatment of tumors. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is a molecule of the invention. The label or package insert indicates that the composition is used for treating the particular condition. The label or package insert will further comprise instructions for administering the composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products.

Additionally, the article of manufacture may further comprise a second container comprising a pharmaceutically-acceptable buffer, such as bacteriostatic water for injection, phosphate-buffered saline, Ringer's solution and dextrose solution. It may further include other materials from a commercial and user standpoint, including other buffers, diluents, filters, needles, and syringes.

The invention is specifically exemplified by the construction and testing of molecules binding to Lewis Y and Her2. The molecules are inducing a strong immediate cytotoxicity to target cells expressing both antigens, whereas neither the parental anti-LewisY antibody nor the parental anti-HER2 Fcab is able to exert such a strong effect without complement or effector cells.

Particularly preferred is a biotherapeutic comprising the antigen-binding sites, or respective sequences of IGN311, a humanized anti-LeY antibody, and a HER2 binder selected from the group consisting of Fcab H10-03-6 and Fcab H561G3M1G4, which are Fc fragments mutagenized in the structural loops of the CH3 domains to create binding sites to HER2, and mutants thereof Multispecific immunoglobulins, including bispecific constructs, based on the CDR sequences of IGN311 and the non-CDR binding sequences of the respective Fcab molecules are particularly preferred.

The invention is further described by the following examples.

### EXAMPLE 1

### Design of Bispecific Antibodies Binding to Lewis Y and HER2

IGN311 is a humanized antibody derived from murine antibody ABL364 (Co et al., 1996, Cancer Res. Volume 56, pages 1118 ff.). In order to provide a HER2-specificity to this antibody, the CH3-domain of the IGN311 molecule is replaced by the CH3 molecule of:

a) the anti-HER2 Fcab 10-03-6 (Wozniak-Knopp et al. Protein Eng Des Sel., 2010 volume 23, pages 289ff) (the composed construct is called “molecule no. 1”)

b) the anti-HER2 Fcab H561G3M1G4 (PCT patent application WO2009/132876) (the composed construct is called “molecule no. 2”).

The sequences of the final mature proteins (without signal peptide) are thus (single letter amino acid code):

a) molecule no. 1 (IGN311/Fcab 10-03-6)

Heavy chain:
SEQ ID NO: 1
EVQLLESGGGLVQPGGSLRLSCAASGFTFSDYYMYWVRQAPEKRLE
WVAYISNGGGSSHYVDSVKGRFTISRDNAKNTLYLQMNSLRAEDTA
LYHCARGMDYGAWFAYWGQGTLVTVSSASTKGPSVFPLAPSSKSTS
GGTAALGCLVKDYFPEPVTVSWNSGALTSGVHTFPAVLQSSGLYSL
SSVVTVPSSSLGTQTYICNVNHKPSNTKVDKKVEPKSCDKTHTCPP
CPAPELLGGPSVFLFPPKPKDTLMISRTPEVTCVVVDVSHEDPEVK
FNWYVDGVEVHNAKTKPREEQYNSTYRVVSVLTVLHQDWLNGKEYK
CKVSNKALPAPIEKTISKAKGQPREPQVYTLPPSRDEYLHGDVSLT
CLVKGFYPSDIAVEWESNGQPENNYKTTPPVLDSDGSFFLYSKLTV
ARYSPRMLRWAHGNVFSCSVMHEALHNHYTQKSLSLSPGK
Light chain:
SEQ ID NO: 2
DIVMTQSPLSLPVTPGEPASISCRSSQSIVHSNGNTYLEWYLQKPG
QSPQLLISKVSNRFSGVPDRFSGSGSGTDFTLKISRVEAEDVGVYY
CFQGSHVPFTFGQGTKLEIKRTVAAPSVFIFPPSDEQLKSGTASVV
CLLNNFYPREAKVQWKVDNALQSGNSQESVTEQDSKDSTYSLSSTL
TLSKADYEKHKVYACEVTHQGLSSPVTKSFNRGEC

b) molecule no. 2 (IGN311/Fcab H561G3M1G4)

Heavy chain:
SEQ ID NO: 3
EVQLLESGGGLVQPGGSLRLSCAASGFTFSDYYMYWVRQAPEKRLEW
VAYISNGGGSSHYVDSVKGRFTISRDNAKNTLYLQMNSLRAEDTALY
HCARGMDYGAWFAYWGQGTLVTVSSASTKGPSVFPLAPSSKSTSGGT
AALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKA
LPAPIEKTISKAKGQPREPQVYTLPPSRDEFFTYWVSLTCLVKGFYP
SDIAVEWESNGQPENNYKTTPPVLDSDGSFFLYSKLTVDRRRWTAGN
VFSCSVMHEALHNHYTQKSLSLSPGK
Light chain:
SEQ ID NO: 4
DIVMTQSPLSLPVTPGEPASISCRSSQSIVHSNGNTYLEWYLQKPGQ
SPQLLISKVSNRFSGVPDRFSGSGSGTDFTLKISRVEAEDVGVYYCF
QGSHVPFTFGQGTKLEIKRTVAAPSVFIFPPSDEQLKSGTASVVCLL
NNFYPREAKVQWKVDNALQSGNSQESVTEQDSKDSTYSLSSTLTLSK
ADYEKHKVYACEVTHQGLSSPVTKSFNRGEC

In order to produce these IgG molecules, standard procedures in moleular cloning are applied. Before back-translation of the protein sequences into DNA, leader sequences are being added at the N-terminus of the protein chains. The immunoglobulin signal peptide sequence MELGLSWIFLLAILKGVQC (SEQ ID NO:5) is fused to the heavy chains and the immunoglobulin signal peptide MDMRVPAQLLGLLLLWLPGAKC (SEQ ID NO:6) is fused to the light chain.

The protein sequences are then reverse transcribed into DNA with codons optimal for expression in mammalian cells. After codon optimization, the genes for the heavy chain and for the light chain are cloned separately into a vector for expression of antibodies in mammalian cells (pCEP4, Invitrogen).

All these steps can be performed according to standard molecular cloning techniques (e.g. Cold Spring Harbour Protocols; http://cshprotocols.cshlp.org/).

The genes cloned into the appropriate vectors (pcDNA) can be ordered at gene-synthesis companies such as Geneart, Germany. Such companies also advise as to codon optimization and optimal arrangement of regulatory sequences such as promoters, transcription start elements, termination sites etc.

Plasmids for heavy and light chains of each antibody are co-transfected into HEK293 freestyle cells by standard techniques and incubated. Growing cells are kept and expanded in Roux bottles, roller bottles and stirrer flasks to produce transiently for prolonged periods (2 weeks) the recombinant antibody (Berntzen et al., 2005, J Immunol Methods, volume 298, 93ff).

The antibodies can be purified from collected supernatants of transiently transfected cells by protein A affinity chromatography according to standard protocols (Sambrook et al; 2001, Molecular Cloning: A Laboratory Manual; ISBN 978-087969577-4).

The purified antibodies can be used for cell binding and cytotoxicity assays.

### EXAMPLE 2

### Cytotoxicity Assay

SKBR3 cells (10⁶) are labelled with 100 microCi of ⁵¹Cr for 1 hour at 37° C.; labelled cells are seeded (10⁴ cells. per well in 50 microlitre) into 96-well flat bottom plates and incubated for 2 hours. Then, samples (bispecific molecules or control proteins; 50 microlitre per well in a dilution series from 1 mM down to 0.1 nM) are added. After incubation of aliquots for various periods of time (10 min to 6 hours) at 37° C., supernatants are harvested, and radioactivity is measured with a gamma-counter. Spontaneous release is defined as the cpm released without addition of the molecules, maximum release is defined as the cpm released by addition of Nonidet P-40. Percentage cytotoxicity is calculated as follows: (sample release−sponaneous release)×100/maximum release−spontaneous release).

The half maximal (50%) immediate cytotoxicity (EC50) is reached for molecule no. 1 at around 1 nM and for molecule no. 2 at around 10 nM. Neither of the individual anti-HER2 Fcabs nor the anti-Lewis Y antibody alone, nor a mixture of the anti-Lewis Y antibody with any one of the Fcabs does significant cell killing in this assay under these conditions.

This was very surprising, because a prior art cross-linking of two antibodies, the Lewis Y specific BR55-2 and an EGFR antibody, merely bound to a breast cancer cell with higher affinity, not killing the cell (Kieber-Emmons, Report Number B222453 to the US Army Medical Research & Materiel Command; 1996; US Army Med Research and Mat Cmd, MCMR-RMI-S [70-1y], ltr 6 Jul. 2000, Ft Detrick, Md.).

### EXAMPLE 3

### EC50 Measurement

SKBR3 cells are cultured in RPMI1640 containing 10% fetal calf serum an 8 mM glutamine. The cells are harvested by trypsination. A total of 1×10⁵ cells in PBS containing 0.1% BSA are incubated with dilutions of the bispecific molecules or control proteins (anti-Lewis Y antibody IGN311 and anti-HER Fcabs respectively) in the range of 1 mM to 0.1 nM and incubated on ice for 20 min. After removal of excess of bispecific molecules or control proteins cells are incubated for 10 min on ice with phycoerythrin-R-coupled polyclonal anti-human FC antibody (Sigma). Measurements are performed on a FACS analyzer (FACS Calibur).

The half maximal staining intensity (EC50) is reached for molecule no. 1 at around 1 nM and for molecule no. 2 at around 10 nM.

### EXAMPLE 4

### Determination of Avidity on Target Cells

Bispecific molecules as well as control molecules (each 0.5 nmol), are labeled in PBS with 100 μCi (1 Ci=37 GBq) of ¹²⁵I in Iodo-Gen (Bio-Rad, 10 μg) coated tubes for 20 min at 4° C. Uncoupled iodine is removed by gel filtration on a PD-10 column (GE Healthcare). About 50% of the radioactivity can be recovered. The specific activity is ranging from about 70 to about 200 μCi/nmol.

To determine maximal immunoreactivity and nonimmunoreactive fraction (N), ¹²⁵I-labeled molecules (20 nCi) are incubated with serial dilutions of freshly harvested SKBR3 cells (0.3-100×10⁶ cells per ml). At the binding plateau (reached with 25×10⁶ cells per ml) the maximum immunoreactivity from a representative experiment is typically around 50%. N is obtained by subtracting the maximum immunoreactivity from 100%. For competition assays, the labeled compounds are incubated with serial dilutions of unlabeled bispecific molecules or control molecules (competitors) and 3×10⁶ target cells cells per ml. Nonspecific binding (NS) is determined with an irrelevant mAb IgG (control molecule) labeled with ¹²⁵I, or by measuring the binding of ¹²⁵I-labeled molecule in excess of competitor. Comparative experiments are performed in triplicate, the same day with the same batch of cells, in V-shaped 96-well plates (final volume 300 μl of PBS supplemented with 1 mg/ml of BSA at 4° C. under agitation for 2.5 h). After centrifugation, the amounts of both free and bound ¹²⁵I is measured as above from an aliquot of the supernatant and of the pellet washed once with ice-cold PBS.

Cell dilution experiments and homogenous competition curves (i.e., competition for cell binding between the labeled and unlabeled forms of the same ligand) are analyzed by a linear equilibrium model. The free (F) and bound (B) radioactivity is corrected by subtracting the N and NS, respectively, and expressed in moles per liter, taking into account the specific activity of each isotopic dilution. The parameters Kd (dissociation equilibrium constant) and R (molarity of binding sites) are fitted to the experimental data by regression of the corrected Scatchard equation (B−NS)/(F−N)=R/Kd−(B−NS)/Kd.

The data can be evaluated according to Terskik et al., 1997, proc. Natl. Acad. Sci. USA, volume 64, pages 1663 ff.

Although embodiments have been described herein, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. Highly avid LeY specific biotherapeutic having at least one further binding site having a different specificity to bind an epitope of a glycosylated cell surface molecule of a tumor cell, which is a recombinant antibody with binding sites in the CDR and in the non-CDR region characterized by an EC50 of less than 1 mM to confer immediate cytotoxicity to said tumor cell.

2. The biotherapeutic of claim 1, which binds to said cell surface molecule by its binding sites to LeY and said epitope.

3. The biotherapeutic of claim 1, wherein said epitope is one of a tyrosine kinase receptor.

4. The biotherapeutic of claim 1, wherein said epitope is one of an erbB molecule, selected from the group consisting of EGFR, HER2, HER3, HER4.

5. The biotherapeutic of claim 1, which has at least three binding sites to said tumor cell.

6. The biotherapeutic of claim 1, wherein said immediate cytotoxicity is determined in an in vitro assay comprising incubating said tumor cell with the biotherapeutic as the only active ingredient.

7. The biotherapeutic of claim 1, which has a low binding affinity to normal cells with a Kd of more than 1 mM.

8. A biotherapeutic comprising antigen-binding sites of IGN311 and a HER2 binder selected from the group consisting of H10-03-6 and H561G3M1G4.

9. A method of producing a biotherapeutic, said method comprising:
a) providing a biological with at least one binding site specifically binding to LeY,
b) engineering a molecule to add at least one further binding site specifically binding to an epitope of a glycosylated cell surface molecule of a tumor cell, wherein said binding sites are designed to confer an avidity with an EC50 of less than 1 mM to kill a tumor cell; and then
c) formulating a pharmaceutically acceptable preparation.
